(19) United States
(12) Patent Application Publication
(10) Pub. No.: US 2025/0299830 A1
(43) Pub. Date: Sep. 25, 2025

(54) SYSTEM AND METHOD FOR GENERATING A CONSOLIDATED HEALTH INDEX SCORE BASED ON PHYSICAL ASSESSMENT PERFORMANCE

(71) Applicant: NeckCare hf., Kopavogur (IS)

(72) Inventors: Jona Marin OLAFSDOTTIR, Kopavogur (IS); Orri GUDMUNDSSON, Kopavogur (IS)

(21) Appl. No.: 19/085,831

(22) Filed: Mar. 20, 2025

Related U.S. Application Data
(60) Provisional application No. 63/568,799, filed on Mar. 22, 2024.

(51) Int. Cl. G16H 50/30; G16H 10/60; G16H 20/30; G16H 40/00

(52) U.S. Cl. CPC G16H 50/30; G16H 10/60; G16H 20/30; G16H 40/00

(57) ABSTRACT

A system and method for enhancing patient education generates a consolidated index score based on a patient's performance in various computer-directed assessments. One or more of these assessments evaluate different aspects of physical health and function, including range of motion, proprioception, and movement control. The system and method may feature a dynamic calculation that adjusts the weighting of a patient's symptoms and elements as needed. The consolidated index score provides a single numerical value that assesses overall physical health and tracks improvement.

### FIELD OF THE DISCLOSURE

The disclosure pertains to a system and method for generating a consolidated health index score based on movements of the musculoskeletal system performed during a computer-assisted physical assessment.

### BACKGROUND

Existing sensorimotor control tests, such as the Butterfly test for assessing musculoskeletal movement, offer adequate diagnostic accuracy for distinguishing between patients with cervical spine impairment and healthy individuals. An exemplary method for the assessment and graded training of sensorimotor functions is detailed in international application No. PCT/IS2010/000010, filed on Jul. 7, 2010, and published as WO 2011/004403 A1 on Jan. 13, 2011, which are incorporated herein by reference.

Current systems and methods for objectively assessing sensorimotor control and other physical functions often generate a wealth of data. The performance results presented to healthcare providers include numerous parameters. The effectiveness of these systems relies on providers to interpret these extensive data sets and parameters to deduce a diagnosis and formulate appropriate treatment based on the results. This demanding analysis and interpretation process is time-consuming and requires specialized provider expertise, which can lead to low adoption rates of new devices and methods in clinical settings, ultimately impeding optimal patient care.

Explaining complex performance results to a layperson can be challenging. As a result, patients often miss receiving effective communication regarding the status of their condition or the rationale behind provided treatment approaches, which may lead to decreased motivation, engagement, and self-advocacy among patients.

Therefore, there is a need for an improved system and method for assessing musculoskeletal movement and effectively communicating such assessments to patients.

### SUMMARY

Solutions are provided herein to address the shortcomings in the prior art by generating a consolidated health index score that may assist healthcare providers in interpreting quantitative assessment results and communicating with the patient regarding condition, status, and progress. For example, after completing multiple assessments, e.g., a range of motion test, a joint position error test, and/or a Butterfly test, the consolidated health index score provides an overall gauge for health conditions. While one physiological function may decline, another can improve simultaneously during treatment/rehabilitation. Actively quantifying the improvement and decline of each physiological function allows patients and providers to focus on improving certain physiological functions while simultaneously maintaining already-improved physiological functions.

A solution is provided to keep patients and providers informed of a condition affecting the musculoskeletal or nervous system of the patient by providing a consolidated health index scoring system and method based on physical assessment performance. The disclosed solution overcomes existing challenges related to patient education. The solution offers an individualized approach by allowing patients to informatively monitor health conditions and actively participate in their healthcare decisions.

The system includes a first computing unit and a tracking device in an embodiment. The tracking device is connected to the first computing unit has an assessment application, at least one processor, one or more hardware storage devices, and a display. The assessment application includes one or more computer-directed assessments. The tracking device is communicatively connected to the first computing unit and configured to observe the physical assessment performance of the individual during one or more computer-directed assessments.

The one or more hardware storage devices of the system store instructions that are configured to execute various commands. The assessment application is configured to initiate a first computer-directed assessment on the assessment application for completion using the tracking device. The assessment application is further configured to obtain the individual's first performance dataset from the tracking device and based on the first computer-directed assessment to produce a first subindex score. The assessment application is configured then to initiate a second computer-directed assessment on the assessment application arranged to be completed using the tracking device. The assessment application is further configured to obtain a second performance dataset of the individual from the tracking device (104, 106) and based on the second computer-directed assessment for producing a second subindex score. Embodiments may include two or more computer-directed assessments to obtain subindex scores. Each subindex score is based on weighted metrics derived from a respective computer-directed assessment.

The assessment application assigns a weighting factor to each of the first and second subindex scores. Because certain metrics may correlate more to certain conditions than others, assigning a weighting factor allows the consolidated health index score to be calculated as a “best” consensus value from multiple assessments. The assessment application is further configured to generate the consolidated health index score for presentation on the display based on, among others, weighting factors with combined first and second subindex scores. In an embodiment, each subindex is generated based on several weighted metrics. Weighting factors may also be assigned to each metric that comprises the respective subindex score. The consolidated health index score may also be calculated by comparing previous individualized or normative data or modulated based on subjective assessments.

While a relatively low consolidated health index score does not necessarily mean a patient is “unhealthy,” it may indicate potential issues with one or more physiological functions. Additionally, rather than providing a self-supervised score for physiological function(s), the consolidated health index score is provided to one or more providers for interacting with and communicating with patients. It is difficult for some patients to understand the many tested variables together and also to understand the variables and quantities that they do not have a sense of, i.e., for many people, being told that one has a maximum neck flexion of 20 degrees does not provide comprehensible or actionable information. In reality, this measurement indicates a rather severe mobility restriction. Because of the difficulty experienced by some patients in understanding and interpreting data about their health, the consolidated health index score assists healthcare providers in communicating a simplified message to the patient regarding their condition, status, and progress.

The computer-directed assessments are configured to derive quantifiable metrics and to evaluate physiological functions such as range of motion, mobility, proprioception, balance, neuromuscular control, sensorimotor control, oculomotor control, coordination, vestibular function, reaction time, strength, or endurance. In a preferred embodiment, the first computer-directed assessment evaluates a first metric or set of metrics different from the second metric or set of metrics evaluated in the second computer-directed assessment. Several metrics can be derived from each computer-directed assessment.

The assessment application is configured to automatically recommend one or more rehabilitation or exercise strategies based on the subindices and consolidated health index score. Such rehabilitation or exercise strategies may be altered based on subsequently executed computer-directed assessments. In an embodiment, the assessment application is arranged to dynamically update one or more rehabilitation or exercise strategies based on the performance of the individual completing one or more computer-directed assessments.

Determining the consolidated health index may include any of the following steps, considered alone or combined. A step of data capture involves capturing objective data with a tracking device during computer-directed assessments. Subjective data and patient characteristics (e.g., physical characteristics, patient history, symptoms, etc.) are captured as user inputs into the system. Another step involves data analysis, whereby the output from the data analysis results from at least one predefined parameter or metric. Each metric is specific to each computer-directed assessment.

According to possible steps to the method, another step may include data transformation. In data transformation, the metrics are transformed using a transfer function. The transfer function is a mathematical function that includes coefficients determined based on a historical dataset from a population (e.g., healthy individuals, patients, etc). The transfer function and coefficients can vary depending on the metric, the assessment, patient characteristics, the set of criteria, or the comparison population used. After data transformation, each metric is represented on a bounded scale (e.g., 0-5, 0-100, −10 to +10, etc.). Another step may result in a subindex calculation. According to the subindex calculation, weights are generated and assigned to each metric. The weights may be determined based on the metric (including type and value), the assessment, patient characteristics, the set of criteria, or the comparison population used. Weighted metrics are combined to form a subindex on a bounded scale for each computer-directed assessment.

According to the method, a consolidated health index calculation may be another step in which weights are assigned to each subindex. The weights may be determined based on the subindex, the assessment, patient characteristics, the set of criteria, or the comparison population used. Weighted subindices are combined to form a consolidated health index represented on a bounded scale.

The method may include optional or further steps that include or exclude subindices based on subjective data and a selection of the type of population used in prior steps (i.e., healthy, patient, athletic, etc.)

An objective of the consolidated health index and subindices is to simplify the presentation and interpretation of physiological assessment results, which often comprise many metrics. Providing a solution that simplifies assessment results benefits healthcare providers and patients and addresses the issues of information overload and provider-patient communication breakdowns.

These and other features, aspects, and advantages of the present disclosure will help better understand the following description, appended claims, and accompanying drawings.

### BRIEF DESCRIPTION OF THE DRAWINGS

To describe how the advantages and features of the systems and methods described herein can be obtained, a more particular description of the embodiments briefly described above will be rendered by reference to specific embodiments thereof, which are illustrated in the appended drawings. Understanding that these drawings depict only typical embodiments of the systems and methods described herein and are not limiting in their scope, certain systems and methods will be described and explained with additional specificity and detail with the accompanying drawings.

FIG. 1 illustrates a system for executing a computer-directed assessment to evaluate a musculoskeletal or neuromuscular health condition.

FIG. 2 illustrates a system of devices and a service for performing the computer-directed assessment.

FIG. 3 illustrates a flowchart of the method for generating a consolidated health index score based on the system's implemented physical assessment performance.

FIG. 4 illustrates an exemplary computer-directed assessment implemented with a graphical user interface.

FIGS. 5-6 illustrate alternative computer-directed assessments implemented with a graphical user interface.

FIG. 7A illustrates an exemplary graphical user interface of a collective index scoring report.

FIG. 7B illustrates another graphical user interface of the collective index scoring report.

FIG. 7C illustrates another graphical user interface of the collective index scoring report.

FIG. 8 illustrates an exemplary graphical user interface of movement control as a subindex classification.

FIG. 9A illustrates an exemplary graphical user interface of range of motion as a subindex classification.

FIG. 9B illustrates another graphical user interface of range of motion as a subindex classification.

FIG. 10 illustrates an exemplary graphical user interface of proprioception as a subindex classification.

FIG. 11 illustrates an exemplary graphical user interface of a dynamic treatment schedule based on results obtained from the computer-directed assessment.

FIG. 12A illustrates an exemplary graphical user interface of a modified index scoring report.

FIG. 12B illustrates another graphical user interface of a modified index scoring report.

FIG. 13 illustrates a flowchart for generating a consolidated health index score based on one or more subindexes obtained from the computer-directed assessment.

The drawing figures are not necessarily drawn to scale. Instead, they are drawn to provide a better understanding of the components and are not intended to be limiting in scope but to provide exemplary illustrations.

### Definitions

For ease of understanding the disclosed embodiments of the present disclosure and associated method and system elements, a description of a few terms may be helpful.

The term ‘computer-directed assessment’ generally refers to an interactive test or exercise (i.e., Butterfly test, range of motion test, relocation test, rehabilitation exercise) conducted using the assessment application performed by an individual, intended to evaluate a certain physical function, wherein the performance of the assessment test may be used in classifying the individual as having an impairment or condition. A computer-directed assessment may be related to motion, strength, balance, etc., and may also be used as a rehabilitative exercise for treating or improving a condition.

The term ‘computer’ or ‘computing unit’ may include any device that comprises at least one processor and that electronically executes one or more programs, such as a user interface program or software program, and may include personal computers, laptop computers, servers, portable media players, handheld devices, cellular phones, microprocessor-based programmable consumer electronic or appliances, and other similar electronic devices that include circuitry for wirelessly sending or receiving information.

The term ‘impairment’ refers to a condition, disability, injury, or symptomatic state affecting an individual's muscular, skeletal, or nervous systems.

The term ‘health index score’ generally refers to a consolidated, numerical value or grade used to quantify the overall status of an individual based on one or more performances by the individual using the computer-directed assessment. In an embodiment, the health index score provides a metric to gauge overall neck health and monitor improvement.

The term ‘individual’ refers to a system user, specifically, a patient or person using the tracking device and undergoing the assessment test.

The term ‘movement control’ refers to sensorimotor control, neuromuscular control, and the like.

Unless otherwise specified, the term ‘network’ refers to one or more data links, e.g., comprising a database, that enable the wired or wireless transport of electronic data between computer systems or modules or other electronic devices.

The term ‘patient education’ generally refers to the process of a healthcare provider explaining to a patient the meaning of assessments/tests results and communicating the status of the patient's condition and progress.

The term ‘processor’ or ‘processing unit’ refers to one or more devices, circuits, or processing cores configured to process data, such as computer program instructions, and includes personal computers, desktop computers, laptop computers, message processors, handheld devices, multi-processor systems, microprocessor-based or programmable consumer electronics, network PCs, minicomputers, mainframe computers, mobile telephones, PDAs, tablets, pagers, routers, switches, and the like. Unless otherwise stated, references to a first processor may also apply to a second processor and vice versa.

The term ‘provider’ may include a medical or healthcare professional (e.g., such as a doctor, a nurse, a physical therapist, chiropractor, and the like), an exercise professional (e.g., such as a coach, an athletic trainer, a nutritionist, and the like). As used herein, and without limiting the foregoing, a ‘healthcare professional’ may be a human being, a robot, a virtual assistant, or an artificially intelligent entity, such entity including a software program, integrated software, and hardware, or hardware alone.

The term ‘service’ refers to an automated program that performs different actions based on input. As used herein, the terms ‘executable module,’ ‘executable component,’ ‘component,’ ‘module,’ ‘service,’ or ‘engine’ can refer to hardware processing units or to software objects, routines, or methods that may be executed on or with the system.

The term ‘software’ generally refers to computer-executable instructions, code, data, applications, programs, program modules, or the like maintained in or on any form or type of computer-readable media that is configured for storing computer-executable instructions or the like in a manner that is accessible to a computing unit.

The term ‘subindex score’ or ‘subindex’ refers to one or more types and values used as input to generate the health index score. A subindex score may quantify different elements of health and function, such as range of motion, proprioception, balance, sensorimotor control, neuromuscular control, strength, oculomotor control, coordination, vestibular function, reaction time, endurance, or cognition, etc. The subindex score may be considered a “consolidated score” because it is, in some cases, based on a combination of weighted metrics derived from the respective computer-directed assessment. “Metric” describes the parameters on which the subindex score is based. For example, a range of motion (ROM) assessment results in, for example, 20 metrics (max flexion is one metric, max extension is another, etc.), which are used to calculate the ROM subindex score. The subindex score preferably has a single numerical value, although it is not limited.

The term ‘tracking device’ refers to an instrument for tracking quantifiable data pertaining to health and function of an individual. As further explained below, the tracking device can include any sensor, camera, or other device capable of tracking movement and location and providing data acquisition that would be understood and available to one having ordinary skill in the art. Non-limiting examples of the tracking device include a headset, camera, IMU, or LiDAR for tracking movement, a force plate for balance, a dynamometer for strength, an eye tracker for eye movement or attention, an electromyography (EMG) or mechanomyogram (MMG) for muscle activity/contraction, an electroencephalogram (EEG) for brain activity or cognition, an electrocardiogram (ECG) for heart activity, a voice recognition unit for cognition, or a wearable device that is communicatively connected to a computing unit of the system.

As used herein, reference to any machine learning or artificial intelligence may include any machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (SVM), artificial intelligence device(s), or any other type of intelligent computing system. Any training data may be used (and perhaps later refined) to train the machine learning algorithm to perform the disclosed operations dynamically.

### DETAILED DESCRIPTION OF VARIOUS EMBODIMENTS

A better understanding of different embodiments of the disclosure may be had from the following description, which is read with the accompanying drawings in which reference characters refer to like elements. While the disclosure is susceptible to various modifications and alternative constructions, certain illustrative embodiments are in the drawings and are described below. It should be understood, however, that there is no intention to limit the disclosure to the embodiments disclosed; on the contrary, the intention covers all modifications, alternative constructions, combinations, and equivalents falling within the spirit and scope of the disclosure.

With respect to the use of plural or singular terms herein, those skilled in the art may translate the terms from the plural to the singular or from the singular to the plural as is appropriate to the context or application. The various singular/plural permutations may be expressly set forth herein for the sake of clarity.

It will be understood that unless a term is defined to possess a described meaning, there is no intent to limit the meaning of such term, either expressly or indirectly, beyond its plain or ordinary meaning.

The disclosed system is based on the functional and statistical interpretation of objective, innovative data derived from measurements of movements, such as musculoskeletal movements of individuals recorded in conjunction with one or more computer-directed assessments. However, some assessments may not involve musculoskeletal movements and may instead involve data derived from strength, cognitive performance, and balance in conjunction with the assessments. The disclosed solution provides a system and method for enhancing patient education by simplifying the interpretation of quantitative assessment data.

Measuring, storing, analyzing, comparing, and classifying diverse musculoskeletal movement variables or other assessment variables gathered with one or more computer-directed assessments allows for automatic treatment recommendation alterations. The consolidated health index score provides invaluable insight and an individualized approach by allowing patients to monitor health conditions informatively and actively participate in their healthcare decisions.

Transforming and applying weighting factors (weight/rank) to the collected performance data to consolidate and simplify the patient's results improves the system to inform patients of possible conditions affecting the musculoskeletal and neuromuscular system and to focus treatment on areas where physical improvement is needed. The weighting factors may be applied to highlight data useful to rehabilitation and other patient improvements.

### Exemplary Embodiment to Objectively Assess and Diagnose Cervical Impairment

Neck pain and neck-related conditions affect nearly two-thirds of the general population at least once in their life and are a growing healthcare concern. Common causes for the related conditions can include whiplash-associated disorder, head impact/concussion, strenuous working conditions, or sustained poor posture. For example, professionals who spend many hours hunched over their workspace—such as surgeons and dentists—frequently develop neck pain. Those who wear heavy protective helmets, including athletes, jet and helicopter pilots, warriors, and firefighters, are also at risk. The neck can also be involved in symptoms and conditions without the presence of neck pain, such as cervicogenic headache and cervicogenic dizziness.

Clinicians currently use techniques to assess neck impairment. Yet, such techniques have significant drawbacks because most of them rely on cumbersome tests, such as manually operated range-of-motion (ROM) tests, making it difficult to gauge the extent of an injury or track progress during therapy. These manually operated ROM tests are also prone to operator error and may lack consistency in implementation and practice. Some techniques to assess proprioception and sensorimotor control also require labor-intensive manual procedures involving a laser pointer attached to the patient's head and error marking or counting by the healthcare provider.

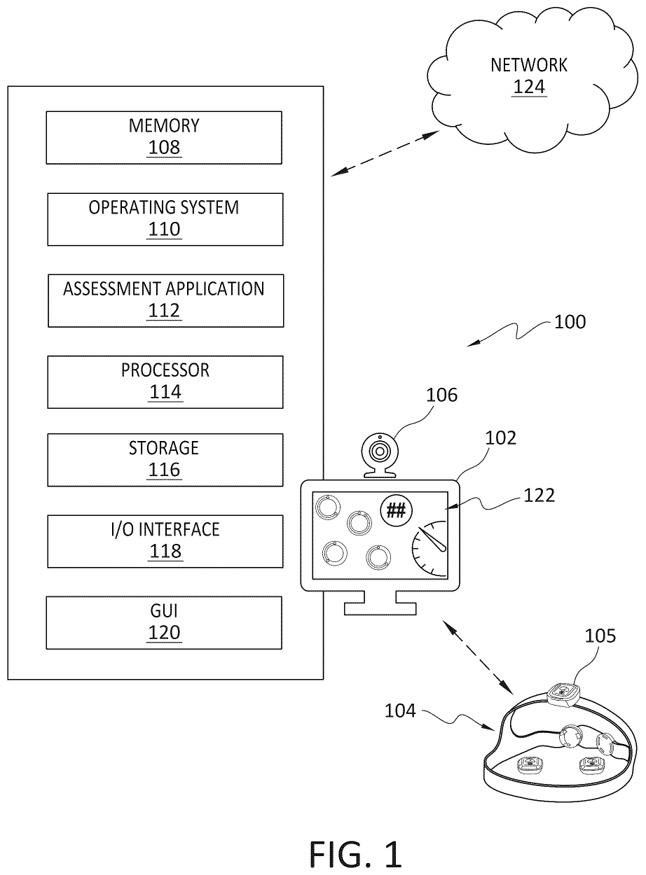

FIG. 1 illustrates an assessment system 100 schematic for evaluating individuals with proprioceptive, mobility, and sensorimotor impairments. The system 100 comprises a computing unit 102 having the disclosed assessment application 112, a tracking device 104, and a secure cloud-based network 124. The assessment application 112 of the system 100 uses data input received from the tracking device 104 to generate a performance dataset based on a computer-directed assessment 136. Various movement data is received from the tracking device 104 and subsequently stored and processed with the computing unit 102 during and after the data capture process. Advantageously, clinicians using the system 100 may remotely track an individual's progress, obviating the reliance on self-reporting to confirm patient compliance.

An example of a tracking device is provided by NeckCare hf of Reykjavik, Iceland, and described as a “NeckCare Device,” and the use of such tracking device is described in U.S. Pat. No. 9,757,055, granted on Sep. 12, 2017, and incorporated herein by reference.

While the above is an exemplary tracking device that uses an inertial measurement unit sensor, the disclosure is not limited to the exemplary tracking device. Rather, a tracking device or a plurality of tracking devices need not be attached to the individual and can include at least one sensor, camera, and/or other device capable of tracking movement and location and providing data acquisition that would be understood and available to one having ordinary skill in the art. Other non-limiting examples may include a force plate for balance, a dynamometer for strength, an eye tracker for eye movement or attention, an electromyography (EMG) or mechanomyogram (MMG) for muscle activity/contraction, an electroencephalogram (EEG) for brain activity or cognition, an electrocardiogram (ECG) for heart activity, and voice recognition unit for cognition.

In an embodiment, the tracking device 104 is a distinct hardware component of the system 100. The tracking device 104 may be a wearable device attached to the individual. For example, tracking device 104 comprises a sensor unit or sensor 105, with one or more inertial measurement units (IMUs) (or similar) attached to an individual. In an embodiment, the tracking device 104 is an adjustable headgear that measures head movement via a Bluetooth-enabled, custom-built sensor 105 that syncs with a computing unit 102 or application 112. The tracking device 104 advantageously features a minimalist design that allows a patient to easily progress through physical therapy without excess weight or resistance aggravating their impairment. In an embodiment, the tracking device 104 weighs less than 55 g, preferably less than 100 g.

In an embodiment, each sensor 105 contains one or more IMUs that report changes in angular position using the IMUs built-in accelerometers and gyroscopes and algorithm. In an embodiment, the tracking device 104 comprises a first sensor 105 attachable to a head, neck, or limb of the human subject and a second sensor (i.e., similar to sensor 105) attachable to a torso or a trunk of the human subject. IMUs of the sensor 105 detect movement in the three cardinal axes and can precisely measure three-dimensional rotational velocity, linear acceleration, and magnetic field.

The assessment application 112 utilizes a sensor fusion algorithm to derive the resultant rotational position (orientation) and minimize drift. In an exemplary embodiment, the sensor fusion algorithm is implemented via a remote processor (e.g., processor 114), which may be embedded in the sensor 105 of the tracking device 104 before communicating the derived data to the assessment application 112 for further processing by a processor 114 on the computing unit 102. Using Bluetooth, the measurements are wirelessly transmitted to a computing unit 102 and, via the assessment application 112, displayed on a monitor or display 122 the head position and movement in real-time. The wireless connection and transmission capability make the system 100 an ideal solution to support patients when practicing physical therapy exercises in the clinic or at home.

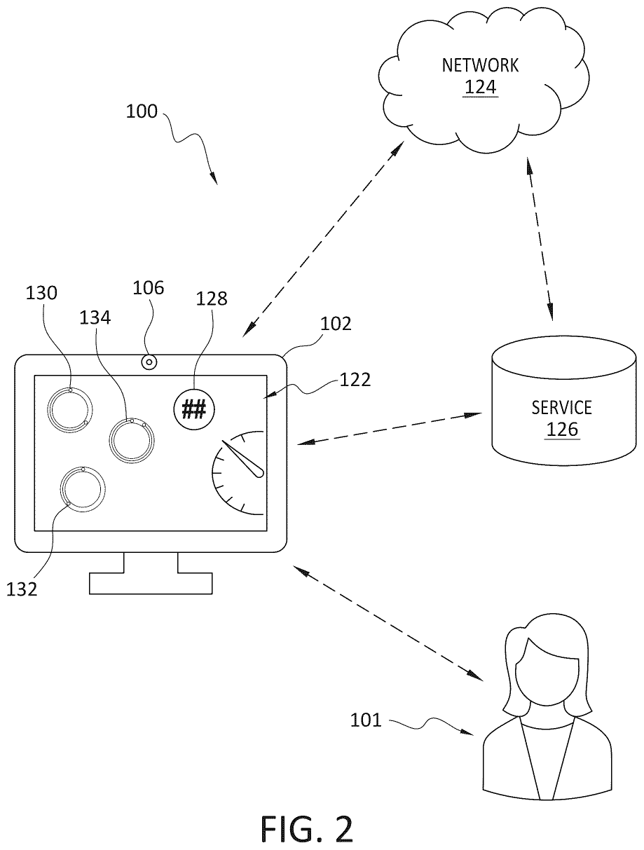

The assessment application 112 processes the measurements from the tracking device 104 and produces quantitative 1D, 2D, or 3D metrics on head-neck movements, i.e., performance datasets. By analyzing the data received from the tracking device 104, it is possible to compare individual results, identify healthy and unhealthy musculoskeletal movements, and generate a consolidated health index score 128 (see FIG. 2) for an individual 101. Advantageously, the assessment application 112 is configured to detect compensative effort or positioning of the patient using the tracking device 104.

In determining the consolidated health index, as described in various aspects below, the method may include any of the following steps, either considered alone or in combination. A step of data capture involves capturing objective data with a tracking device during computer-directed assessments. In an embodiment, a secondary device is combined with the tracking device to assess, wherein an input field is provided to enter measurements related to the secondary device, and said measurements are subsequently used for index classification. For example, if strength was to be assessed with the secondary device (e.g., weights, dynamometers (hand-held or fixed), strain gauges, force platforms) in combination with the tracking device, the measurements from the secondary device are recorded in the input field and then entered into the system for index calculation. Subjective data and patient characteristics (e.g., physical characteristics, patient history, symptoms, etc.) are captured as user inputs into the system. Another step involves data analysis, whereby the output from the data analysis results from at least one predefined parameter or metric. Each metric or set of metrics is specific to each computer-directed assessment.

According to possible steps to the method, another step includes data transformation. In data transformation, the metrics are transformed using a transfer function. The transfer function is a mathematical function that includes coefficients determined based on a historical dataset from a population (e.g., healthy population, patient population, etc). The transfer function and coefficients can vary depending on the metric, the assessment, patient characteristics, the set of criteria, or the comparison population used. After data transformation, each metric is represented on a bounded scale (e.g., 0-5, 0-100, −10-10 etc). Another step may result in a subindex calculation. According to the subindex calculation, weights are generated and assigned to each metric. The weights may be determined based on the metric (including type and value), the assessment, patient characteristics, the set of criteria, or the comparison population used. Weighted metrics are combined to form a subindex represented on a bounded scale for each computer-directed assessment.

According to the method, a consolidated health index calculation may yet be another step wherein weights are assigned to each subindex. The weights may be determined based on the subindex, the assessment, patient characteristics, the set of criteria, or the comparison population used. Weighted subindices are combined to form a consolidated health index represented on a bounded scale. Each subindex is generated based on several weighted metrics.

The method may include optional or further steps that include or exclude subindices based on subject data and a selection of the type of population used in prior steps (i.e., healthy, patient, athletic, etc.)

An objective of the consolidated health index and subindices is to simplify the presentation and interpretation of physiological assessment results, which often comprise many metrics or metrics that are not intuitive for a layperson to interpret. Providing a solution that simplifies assessment results benefits healthcare providers and patients and addresses the issues of information overload and provider-patient communication breakdowns.

Subjective data can be inputted by typing information on a keyboard or using a mouse to click and select certain provided data/information/options. For instance, the user can type in the patient age, gender, weight, symptoms, pain level, history, etc. Alternatively, another type of subjective data can be produced from questionnaires (some clinically validated) where a patient answers specific questions related to their condition and where answers are provided using a psychometric response scale, e.g., a Likert scale.

For example, one subindex can be based on pain level; another subindex can be based on a Dizziness Handicap Inventory questionnaire. These can be weighted and combined with the weighted subindices based on objective data. If a patient scores high on all objective assessments but low on subjective assessments, their overall consolidated health index will be lower than if only objective assessments were included.

For factoring of patient characteristic data, the population data can be used during data transformation and will be different for a young person vs. an older person. Weights applied to metrics and subindices may be different for male or female. More importantly, the weights may vary depending on the symptom information provided.

The consolidated health index score 128 may include a representation of subjective assessments (pain scale, questionnaires), either with subindex scores generated based on subjective assessments or by using results from subjective assessments to modulate the index generated based on objective assessments. In an embodiment, an individual may choose whether the consolidated health index score 128 is based only on the objective assessment (i.e., computer-directed assessment) or the combination of objective and subjective assessments.

The technology of the assessment application 112 can be described as a cloud-based Internet of Healthcare Things (IOHT) application connected to a wired or non-wired device. The wired or wireless recording devices may include the following: computerized tomography (CT-Scan); computers of various sizes and operational capabilities; high-energy electromagnetic radiation X-ray; inertial measurement unit (IMU); IT tablets of all shapes, sizes and computing power; microwave diathermy; mobile phones; smart watches; optical sensors; radar operated devices using radio waves to determine the distance angle and radial velocity of individuals; thermal imaging; and video cameras or devices that are capable of recording musculoskeletal movements of individuals.

The computing unit 102 includes a memory 108, operating system 110, assessment application 112, one or more processor(s) 114, storage 116, input-output interface 118, graphical user interface 120, and display 122. The computing unit 102 may represent any suitable type of computer, computing system, server, disk array, or programmable devices such as a handheld device, a networked device, or an embedded device, etc. The computing unit 102 may communicate with one or more networked computers via one or more networks 124, such as a cluster or other distributed computing system, through the I/O interface 118. The I/O interface 118 is configured to transmit data between the computing unit 102 and the tracking device 104, e.g., via wired or wireless connection.

The processor 114 may include one or more devices selected from processors, micro-controllers, digital signal processors, microcomputers, central processing units, field programmable gate arrays, programmable logic devices (i.e., digital logic circuitry), state machines, logic circuits, analog circuits, digital circuits, or any other devices that manipulate signals (analog or digital) based on operational instructions that are stored in the memory 108. Memory 108 may be a single memory device or a plurality of memory devices including but not limited to read-only memory (ROM), random access memory (RAM), volatile memory, non-volatile memory, static random-access memory (SRAM), dynamic random-access memory (DRAM), flash memory, or cache memory. Memory 108 may also include a mass storage device such as a hard drive, optical drive, tape drive, non-volatile solid-state device, or any other device capable of storing digital information.

The processor 114 may operate under the control of an operating system 110 that resides in memory 108. The operating system 110 may manage computer resources so that computer program code embodied as one or more computer programs, such as the assessment application 112 communicatively connected to memory 108 may have instructions executed by the processor 114. In an alternative embodiment, the processor 114 may execute the assessment application 112 directly, in which case the operating system 110 may be omitted.

The computer storage 116 typically includes at least one hard disk drive and may be located externally to the computing unit 102, such as in a separate enclosure or one or more networked computers, one or more networked storage devices (including, for example, a tape or optical drive), and/or one or more other networked devices (including, for example, a server). The storage 116 may also host one or more databases communicatively connected to data collected and received in association with the assessment application 112, wherein the storage 116 includes a cloud-based secured HIPAA-compliant database. The storage 116 may be included in the computing unit 102 and/or in a cloud-based network 124 (or service 126).

The graphical user interface 120 may be operatively coupled to the processor 114 of computing unit 102 in a known manner to allow a system operator to interact directly with the computing unit 102. The graphical user interface 120 may include or be communicatively connected to output devices (e.g., display 122) such as video and/or alphanumeric displays, a touch screen, a speaker, and any other suitable audio and visual indicators capable of providing information to the system operator. The graphical user interface 120 may also include input devices (e.g., tracking device 104) and controls such as an alphanumeric keyboard, a pointing device, keypads, pushbuttons, control knobs, microphones, etc., capable of accepting commands or input from the operator and transmitting the entered input to the processor 114.

In a preferred embodiment, the computing unit 102 is communicatively connected to an integrated tracking device 106, e.g., camera or LiDAR, for observing the movement and positional performance of an individual 101 and/or tracking device 104. The tracking device 106 may be directly and operatively connected to the computing unit 102 and arranged to monitor the individual 101 without being directly attached to the individual.

Those skilled in the art will recognize that the computing environment illustrated in FIG. 1 is not intended to limit the solution(s) in the present disclosure. In addition, various program code described herein may be identified based upon the application or software component within which it is implemented in a specific embodiment of the invention. However, it should be appreciated that any program or hardware nomenclature that follows is used merely for convenience, and thus the disclosed system and method should not be limited to use solely in any specific application identified and/or implied by such nomenclature. It should be further appreciated that the various features, applications, and devices disclosed herein may also be used alone or in any combination. Moreover, given the typically endless number of ways in which computer programs may be organized into routines, procedures, methods, modules, objects, and the like, as well as the various ways in which program functionality may be allocated among various software layers that are resident within a typical computing system (e.g., operating systems, libraries, APIs, applications, applets, etc.), and/or across one or more hardware platforms, it should be appreciated that the invention is not limited to the specific organization and allocation of program or hardware functionality described herein.

FIG. 2 illustrates the system 100 further including a service 126 being communicatively coupled to the computing unit 102 and network 124. In some cases, the service 126 can be a deterministic service that operates fully given a set of inputs without a randomization factor. In other cases, service 126 can be or can include a machine learning (ML) or artificial intelligence engine to enable the service 126 to operate even when faced with a randomization factor. In some implementations, the service 126 is a cloud service operating in a cloud environment. In some implementations, the service 126 is a local service operating on a local device. In some implementations, the service 126 is a hybrid service that includes a cloud component operating in the cloud and a local component operating on a local device. These two components can communicate with one another. The service 126 is generally tasked with operatively connecting the computing unit 102 with the network 124 or another computer. In an embodiment, the service 126 functions as a proxy server or intermediary between the computing unit 102 and the network 124.

In an alternative embodiment, the service 126 functions as a virtual machine. The service 126 may receive incoming data, i.e., a software update, from a network 124. The service 126 may be configured as a collective database storing (e.g., via storage 116) previously obtained data (e.g., historical performance datasets) based on performance datasets of the individuals and/or normative data of a representative population. In other words, first and second subindex scores 130, 132 can be produced using historical performance datasets stored in a collective database 126 and factored into a dynamic calculation with the first and second performance datasets of the individual 101, the collective database 126 being communicatively connected to the first computing unit 102. The historical performance dataset is preferably collected from a group of individuals (population). Historical performance data from the respective individual may also be used in the dynamic calculation together with a population historical dataset. Additionally, patient characteristics might be factored into the dynamic calculation, including parameters such as age, gender, weight, etc.

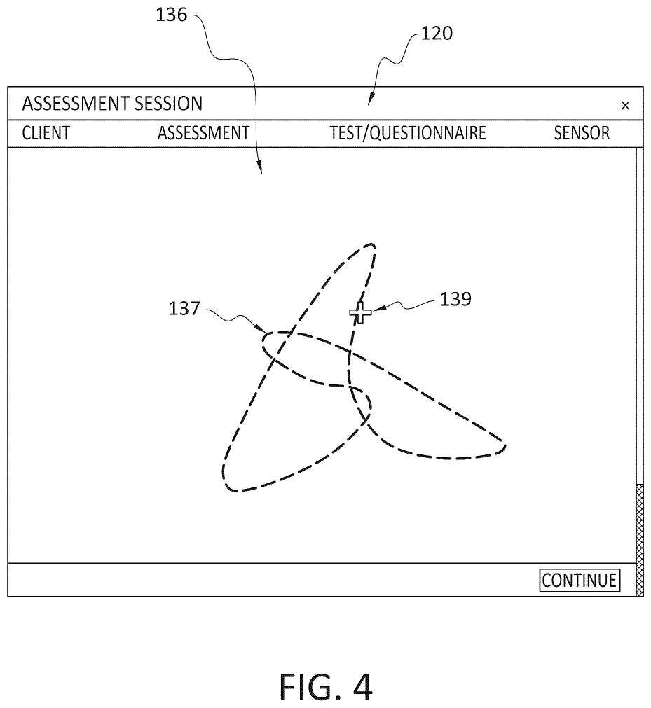

FIG. 2 further illustrates the general architecture and testing setup for the system 100. The tracking device 106, e.g., camera or motion capture device, is integrated with the computing unit 102 and arranged to monitor and record an individual 101 during a computer-directed assessment 136, as shown in FIG. 4. The computing unit 102 collects movement data from the tracking device 104 during the computer-directed assessment 136. In an embodiment, the assessment application 112 of the system 100 is configured to initiate a first computer-directed assessment 136 on the assessment application 112 and obtain a first performance dataset of the individual 101 based on the first computer-directed assessment 136 for producing a first subindex score 130. Subsequently, the assessment application 112 initiates a second computer-directed assessment 138 on the assessment application 112 and obtains a second performance dataset of the individual 101 based on the second computer-directed assessment 138 for producing a second subindex score 132.

A weighting factor is assigned via assessment application 112 to the first and second subindex scores 130, 132. The weighting factor of the first subindex score of 130 may be greater than or equal to or smaller than the weighting factor of the second subindex score 132. Each subindex score is generated based on a combination of metrics or metric scores with different weighting factors. Each subindex score is thus generated from a set of weighted metrics. Thus, in other words, weighting factors comprise two layers of (1) weights for each metric where each subindex is generated from a set of metrics, and (2) weights for each subindex. The weighting factors in each layer may be modulated based on various criteria (i.e., based on certain numerical values of certain metrics, etc.) or a set of rules or conditions, i.e., patient characteristics, symptoms, patient history, progress, or combination of results from the computer-directed assessments.

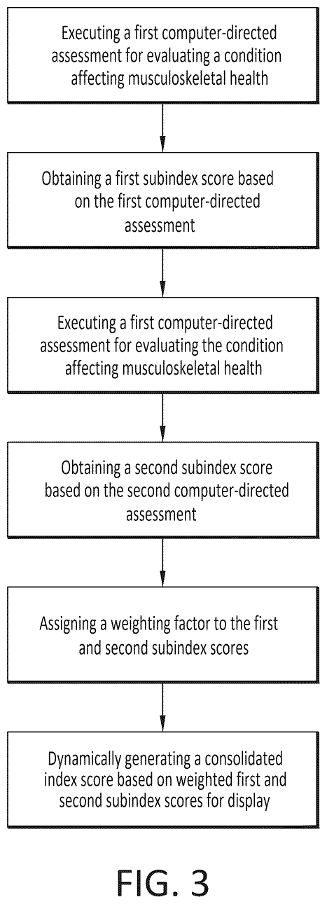

The assessment application 112 automatically generates a consolidated health index score 128 based on the weighting factors and combined first and second subindex scores 130, 132. A flowchart of this process can be observed in FIG. 3. The graphical user interface 120 connected to the assessment application 112 is further configured to present via display 122 the consolidated health index score 128 to the individual 101 in the form of a single numerical value which gauges overall neck health.

FIGS. 4-6 illustrate exemplary computer-directed assessments by the disclosed system, though computer-directed assessments in the claims are not limited to the computer-directed assessments in the drawings. The assessment application 112 of the system 100 preferably comprises multiple computer-directed assessments 136, 138, 140. These computer-directed assessments are arranged to evaluate physiological function, including range of motion, proprioception, balance, neuromuscular control, and strength. Other functions evaluated may include oculomotor control, eye-head-neck coordination, eye-head-limb coordination, gait, reflexes or reaction time, vestibular function, cognitive performance, sleep, endurance, and the like. In a preferred embodiment, more than one computer-directed assessment is used to evaluate different function-based metrics, i.e., a first computer-directed assessment is used to evaluate a first metric or set of metrics that is different from a second metric or set of metrics evaluated with a second computer-directed assessment. For example, a first computer-directed assessment may assess a first metric or set of metrics directed to a range of motion. In contrast, a second computer-directed assessment is then used to assess a second metric or set of metrics directed to proprioception.

FIG. 4 illustrates a first computer-directed assessment 136 designed to evaluate sensorimotor control. The first computer-directed assessment 136 includes a guiding interface 137 for testing certain elements of sensorimotor control, which prompts the user to conduct the sensorimotor control test. The guiding interface 137 may be a moving target, computer-generated path, or another interface element to map a cursor 139 and the corresponding movement of the tracking device 104. In an embodiment, the first computer-directed assessment 136 instructs the individual 101 to follow a computer-generated path (e.g., guiding interface 137) with the tracing cursor (e.g., cursor 139) by moving the head, neck, or limb of the subject, wherein the computer-generated path 137 is presented on the display 122 (e.g., monitor) either as a visible path or a target that moves along an invisible path. In an embodiment, the assessment application 112 records a first performance dataset based on a trajectory path of the cursor 139, and a correlation and/or deviation between the trajectory path of the cursor 139 and the computer-generated path 137. The correlation and/or deviation can further be analyzed using movement parameters (metrics).

During the first computer-directed assessment 136, the assessment application 112 records rotational data at a predefined frequency (Hz), derived from angular velocity and acceleration, etc., of the tracking device 104. This recorded movement data is stored and processed using data analysis and machine learning algorithms by means of statistical and machine learning techniques (e.g., via deep artificial neural network) that uses movement data and derived parameters (metrics) to enhance the accuracy and quality of the assessment and recommended exercises.

During the first computer-directed assessment 136, the assessment application 112 evaluates an individual's ability to control head and neck movements while following the guiding interface 137 in the defined sensorimotor control test. In a particular assessment, the assessment application 112 projects the rotational displacement captured by the tracking device 104 onto the 2D plane coinciding with the screen surface of the display 122 (e.g., a monitor). Using this projection, the assessment application 112 can then compare the path of the guiding interface 137 with the path traced by the cursor 139 that corresponds to the individual's movement.

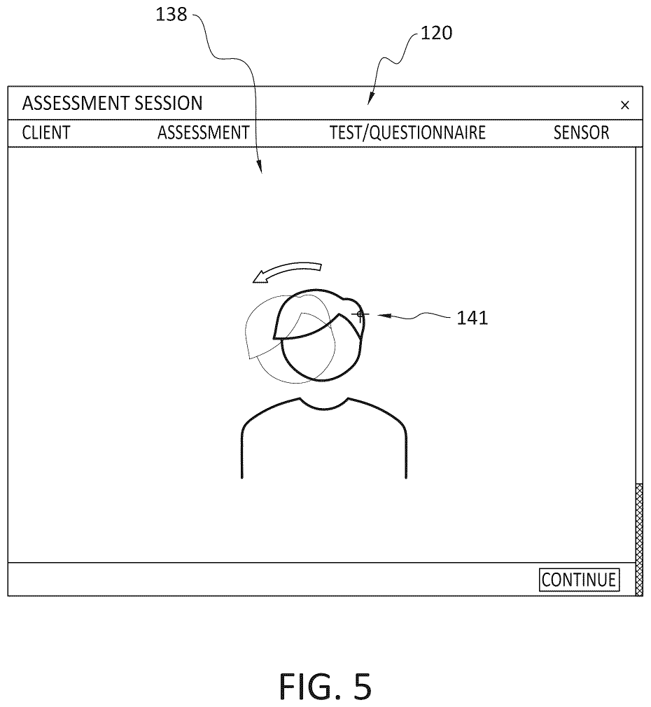

FIG. 5 illustrates a second computer-directed assessment 138 based on the range of motion. The second computer-directed assessment 138 includes an instructional interface 141 for completing the second computer-directed assessment 138. The instructional interface 141 may be configured to prompt an individual to perform tests related to backward, forward, and lateral bending, as well as left and right rotation.

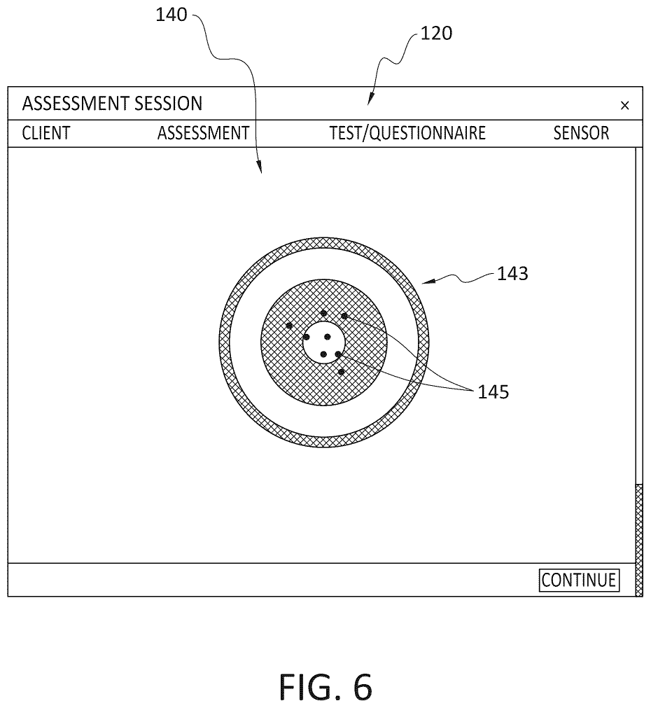

FIG. 6 illustrates a third computer-directed assessment 140 based on proprioception. The third computer-directed assessment 140 includes a positional awareness interface 143, which may include visible and/or hidden graphics from the individual 101 during the execution of the third computer-directed assessment 140, and target marks 145 for recording relocation accuracy. The three computer-directed assessments 136, 138, 140 may be configured as tests, assessments, rehabilitative exercises, and/or treatment tasks.

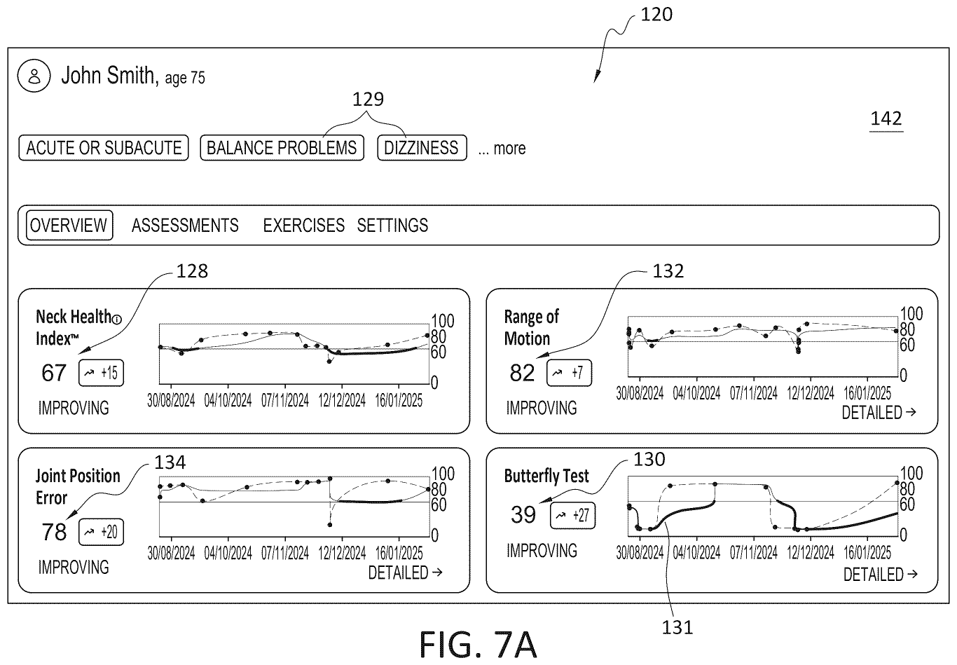

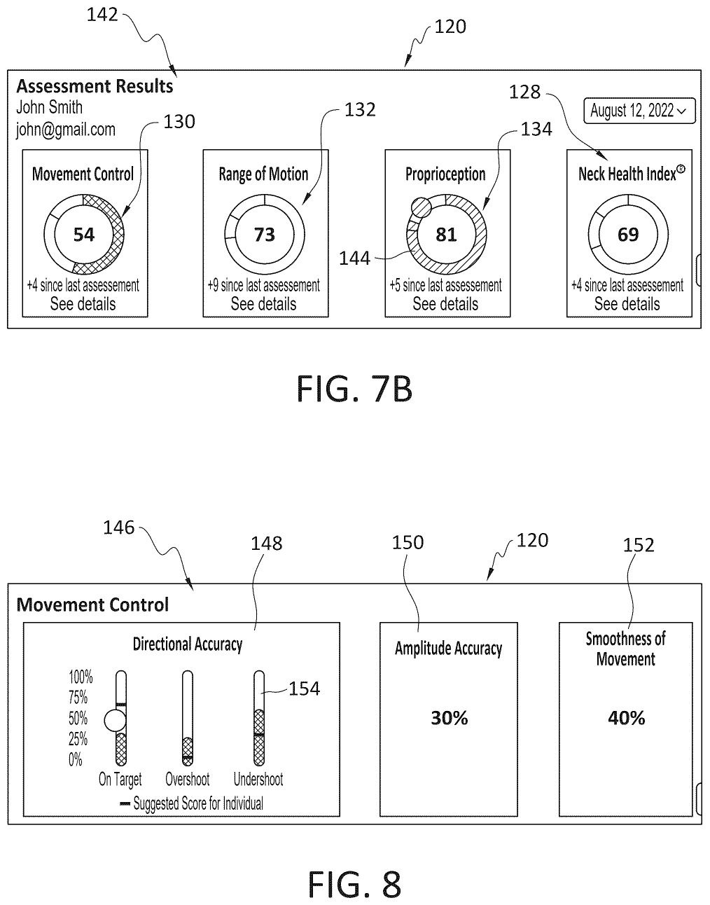

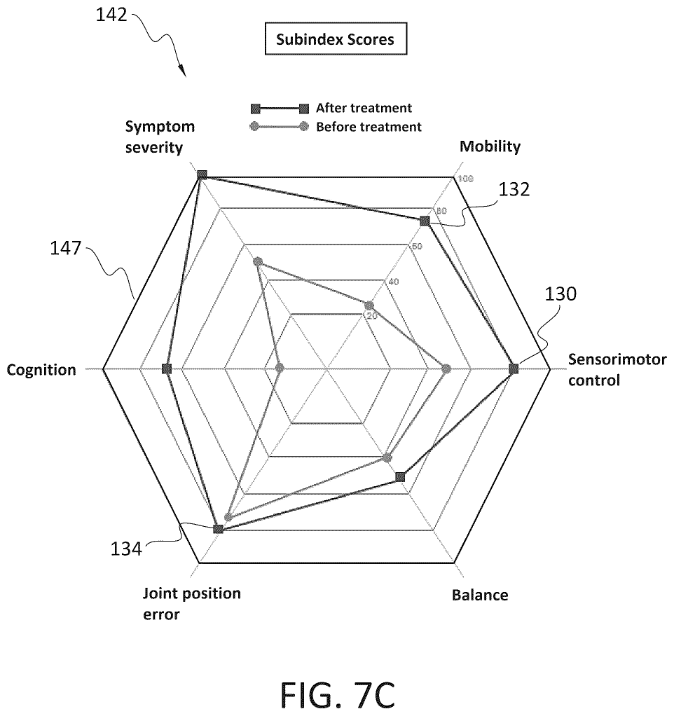

FIG. 7A illustrates the graphical user interface 120 of the system 100 displaying a collective index scoring report 142. The collective index scoring report 142 includes a compilation of subindex scores 130, 132, 134 and provides the consolidated health index score 128 based on the computer-directed assessments 136, 138, 140. Each subindex score 130, 132, 134, and consolidated index score 128 may include a graphical depiction 131 to show how the subindices and consolidated index score change over time for a particular patient. The graphical depiction 131 can illustrate the most recent subindices and the indices over time and/or trends in subindices (e.g., moving average). In an embodiment, the graphical user interface 120 also displays one or more graphical markers 129 to indicate symptoms and/or conditions of the particular patient. The graphical markers 129 may be displayed in order of significance and can be automatically or manually updated over time.

FIG. 7B illustrates another embodiment of the graphical user interface 120 of the system 100 displaying the collective index scoring report 142. The collective index scoring report 142 may include a compilation of subindex scores 130, 132, 134 based on the computer-directed assessments 136, 138, 140 to display an itemized assessment based on physical assessment performance of the individual 101. Each subindex score may include a scoring icon 144 used to convey the result of the computer-directed assessment. For example, the scoring icon 144 may indicate a suboptimal performance based on a computer-direct assessment using a negatively associated design, i.e., color-coded indicator in yellow or red. Alternatively, the scoring icon 144 may indicate an optimal performance based on a computer-direct assessment using a positively associated design, i.e., color-coded indicator in green.

FIG. 7C illustrates another embodiment of the graphical user interface 120 of the system 100 displaying the collective index scoring report 142. In this embodiment, the collective index scoring report 142 presents various subindex scores 130, 132, 134 on a radial plot 147 before and after treatment. The radial plot 147 includes reference to various physiological functions and conditions, including mobility, sensorimotor control, balance, joint position error, cognition, and symptom severity; however, one skilled in the art will recognize that other physiological functions and conditions are contemplated. The radial plot 147 provides an overall improved visualization for the individual 101 to simultaneously observes areas of improvement, and/or areas of regression, relative to other areas during treatment. The radial plot 147 also presents a compact visualization of the data, highlighting relationships between physiological functions and facilitating comparisons, especially for cyclical or multivariate data.

In an embodiment, scores may be provided for subindex parameters (i.e., classifications, indicators, or grades) to show specific results for each metric (e.g., forward bending is 82 out of 100 instead of 48°). Thus, an individual or provider may easily identify areas experiencing greater impairment, and rehabilitation exercises, e.g., automatically generated by the assessment application 112, may highlight parameters needing improvement and attention.

FIG. 8 illustrates the graphical user interface 120 of the system 100 displaying a first subindex report 146. As depicted, the first subindex classification report 146 displays an overview of the first performance dataset contributing to the first subindex score 130. For example, regarding the first computer-directed assessment 136 based on movement control, one or more movement control metrics or indicators 148, 150, 152 are itemized to show performance with respect to each indicator, e.g., using indicator icons 154. A first indicator of movement control indicator 148 may be directed to directional accuracy, wherein metrics relating to ‘on target,’ ‘overshoot,’ and ‘undershoot’ contribute to the first subindex score 130. In other words, the system 100 computes “time-on-target,” which is the percentage of time the cursor 139 is on or in close vicinity of the guiding interface 137 (“target”), wherein the undershoots and overshoots include the proportion of time spent behind or ahead of the target, respectively.

A second movement control indicator 150 may be directed to amplitude accuracy. Amplitude accuracy may be defined as an average difference between the target dot and the subject-control cursor during the first computer-directed assessment 136. A third movement control indicator 152 may relate to smoothness of movement. The smoothness of movement may be defined as a parameter that quantifies jerkiness based on the integration of the quadratic sum of the third derivative of spatial coordinates traced by the subject (e.g., using the tracking device 104) normalized against the same quantity traced by the target. The amplitude accuracy and smoothness of movement metrics may contribute to the first subindex score 130 or provide a similar subindex score.

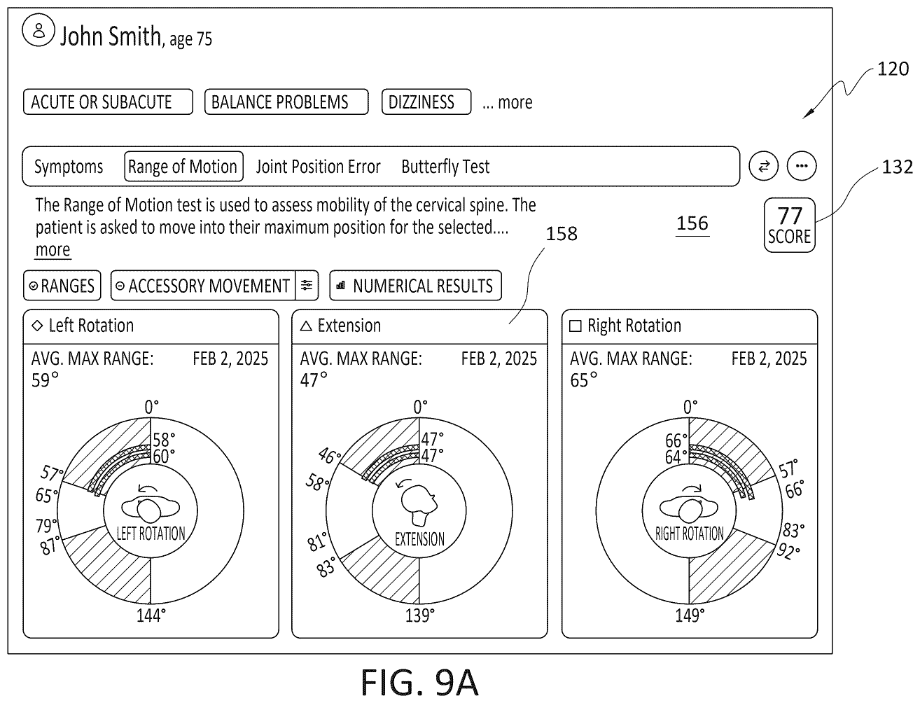

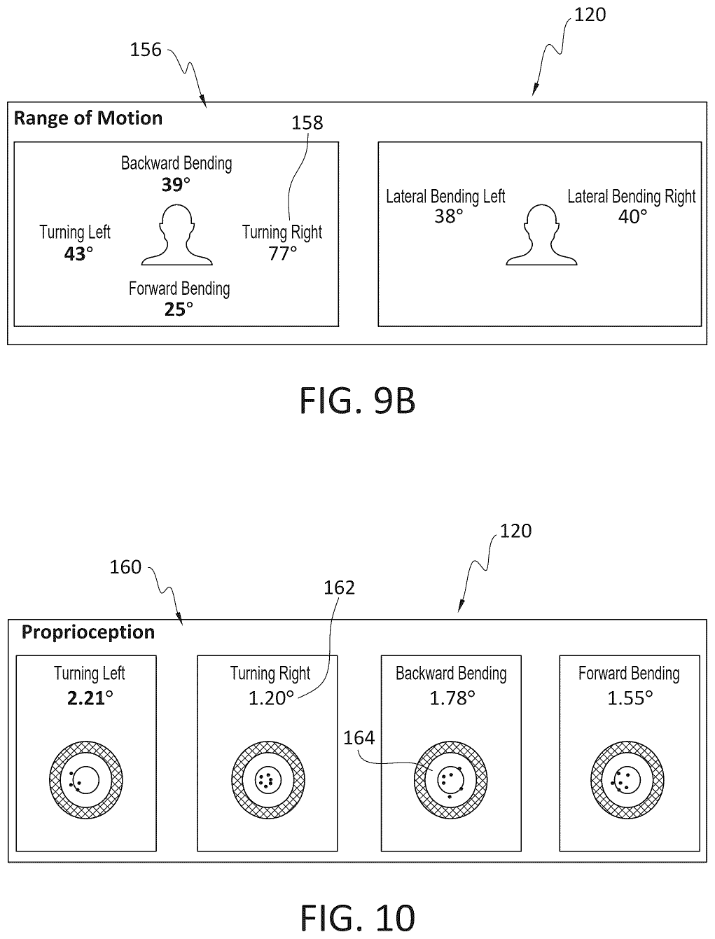

FIG. 9A illustrates the graphical user interface 120 of the system 100 displaying a subindex classification report 156 of an assessment, e.g., for a range of motion. The subindex classification report 156 displays an overview of the performance dataset contributing to the subindex score 132. One or more metric 158 can be displayed with an icon that illustrates a corresponding activity or performance with the recorded measurements or values (e.g., numerical ranges or results).

FIG. 9B illustrates another embodiment of the graphical user interface 120 of the system 100 displaying the second subindex classification report 156. As depicted, the second subindex classification report 156 displays an overview of the second performance dataset contributing to the second subindex score 132. For example, regarding the second computer-directed assessment 138 based on range of motion, one or more range of motion metrics 158 are characterized to show performance concerning various neck movements. The one or more range of motion metrics 158 may relate to backward, forward, lateral bending, and left and right rotation.

FIG. 10 illustrates the graphical user interface 120 of the system 100 displaying a third subindex classification report 160. As depicted, the third subindex classification report 160 displays an overview of the third performance dataset contributing to the third subindex score 134. For example, regarding the third computer-directed assessment 140 based on proprioception, one or more joint position error metrics 162 are characterized to evaluate proprioceptive ability concerning bending and rotation of the neck. The one or more joint position error metrics 162 may relate to finding a neutral posture after backward and forward bending, as well as left and right rotation. Accuracy of the joint position error metrics 162 may be presented as icons 164, similar to the positional awareness interface 143.

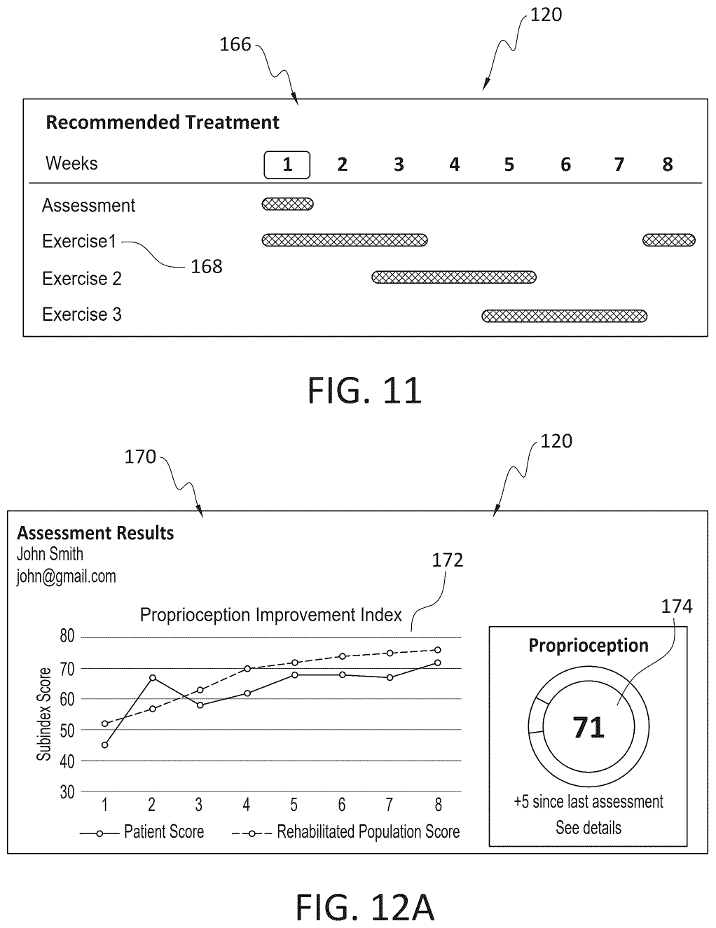

FIG. 11 illustrates the graphical user interface 120 of the system 100 displaying a dynamic treatment schedule or treatment plan 166 specifically tailored to the individual 101. One or more exercise strategies 168 may be recommended or assigned based on one or more computer-directed assessments 136, one or more subindex scores, and/or the consolidated health index score 128. For example, if a suboptimal performance is recorded during the first computer-directed assessment 136 and an optimal performance is recorded during the second computer-directed assessment 138, the assessment application 112 may automatically recommend one or more exercises having a focus on improving physical performance associated with the assessment performance metric(s) evaluated by the first computer-directed assessment 136. In an embodiment, the assessment application 112 may include a deep artificial neural network (ANN) to automatically recommend one or more rehabilitation or exercise strategies 168 based on the consolidated health index score 128, including subindex scores and metrics from each completed computer-directed assessment. The assessment application 112 may be arranged to dynamically adjust and update the at least one recommended rehabilitation or exercise strategies 168 based on the performance of the individual during and or after completion of one or more computer-directed assessments 136, 138 during the rehabilitation or training period. An initial exercise strategy 168 may be generated based on the first time one or more computer-directed assessments are completed. The exercise strategy 168 is then updated after a second time a one or more computer-directed assessments are complete. The first and second times one or more computer-directed assessments are completed typically occur one or more days apart during the rehabilitation or training period to monitor progress.

For individuals performing the computer-directed assessments and receiving regular assessments by a physical therapist, the rehabilitation progress of the individual can be monitored both by the physical therapist and assessment application 112. The rehabilitation strategy may be automatically adjusted according to the individual's performance improvement during each subsequent computer-directed assessment (or rehabilitative exercise).

The assessment application 112 provides a novel method for the evaluation and recommendation of rehabilitation approaches based on the patient's performance data. During an individual's rehabilitation process, there may be a need to improve movement control, proprioception, range of motion, balance, strength, or other physical functions. The system 100 offers exercises via the assessment application 112 that train these functions, e.g., of the neck. The proposed assessment application 112 helps in determining the right type of exercises, difficulty- and intensity levels for each individual, taking into account some or all of the following: (1) the clinical assessment by a healthcare provider, (2) the computer-directed assessments using the system 100, (3) the computer-directed exercises using the system, and (4) the patient's reported data, such as responses to a validated Neck disability index questionnaire and/or pain rating. In an embodiment, a future exercise may be modified (i.e., difficulty level, etc.) based on progress evaluated via exercise performance, e.g., in situations where the patient is improving, as noted by his performance on the exercise, but a re-evaluation assessment has not yet been conducted. By providing a first classification of a patient's rehabilitation needs and an individual therapy plan that will complement other therapy prescribed by an attending healthcare provider, the proposed assessment application will include an artificial-intelligence-based recommendation engine for the (semi)-automatic, individual, and adaptive rehabilitation plan creation based on patient data and changes in the data that can be observed during the patient's rehabilitation. The computer-generated rehabilitation plan may be manually adjusted by the healthcare provider based on their clinical judgment.

Another aspect of the present disclosure relates to a rehabilitation or modified index score 174 of the assessment application 112. In certain embodiments, the consolidated health index score 128 is generated based on comparisons with healthy individuals. However, it may be clinically relevant to also compare computer-directed assessment results of an individual to pathological/symptomatic individuals. For example, it may be unlikely for some individuals to ever reach a normal or “healthy” condition. Providing a modified index score 174 can give an idea of how the individual stands compared to other pathological/symptomatic individuals or rehabilitated individuals. The modified index score 174 may be presented as a subindex score related to a physiological function (e.g., proprioception), as shown, and/or as a consolidated index score like health index score 128. In an embodiment, scores may be provided for subindex metrics (i.e., classifications, indicators, or grades) to show specific results for each metric (e.g., forward bending is 82 out of 100 instead of 48°).

The performance of rehabilitated individuals may be different (likely worse and with different characteristics) from healthy individuals. An unmodified consolidated health index score 128 could thus result in consistently low values if a person realistically cannot reach a normal or healthy baseline. Basing the modified index score 174 on data from pathological/symptomatic or rehabilitated individuals provides comparatively higher scores, which avoids discouraging an individual by comparison to the normal (healthy) baseline. The individualized approach of using a modified index score provides tailored feedback and exercise strategies 168 that would otherwise not be present in a one-size-fits-all approach.

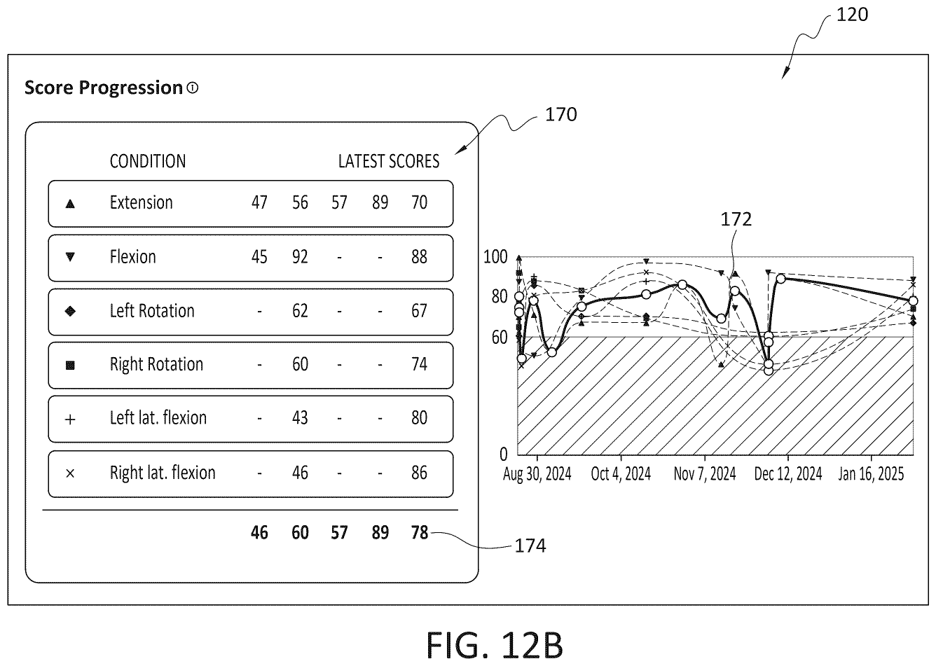

In an embodiment, as depicted in FIG. 12A, the assessment application 112 generates a scoring report 170 having a comparative progression indicator 172. The comparative progression indicator 172 may be provided as a chart or graph and a numerical score to visualize and grade improvement (or decline) of an individual participating in one or more rehabilitation or exercise strategies 168. The comparative progression indicator 172 aids in the assessment of the rate of progression to normal relative to some (e.g., rehabilitated) population. The comparative progression indicator advantageously allows providers and individuals to know how well the individual receiving rehabilitation or training is progressing.

In an embodiment, the modified index score 174 is generated based on the patient's computer-directed assessment history, computer-directed exercise history, rehabilitation duration, and potential rehab structure, and compared to the progression data (i.e., historical performance data) of rehabilitated individuals. The term ‘rehabilitated individual’ generally refers to an individual having gone through rehabilitation (or some other type of therapy) that has progressed from a level of impairment to the highest level of function (e.g., of the neck or limb) possible. As depicted, the comparative progression indicator 172 shows results of an individual compared to a population of rehabilitated individuals for each week a computer-directed assessment was conducted. The modified index scoring report 170 thus provides the patient with a score that represents how well said patient is progressing compared to others.

On the result page for each assessment, various sub-subindices that make up the subindex/score can be presented in a table and/or graphical format to demonstrate how each sub-subindex changes over time between assessments. For example, as illustrated in FIG. 12B, the graphical user interface 120 depicts the scoring report 170 with a plurality of modified index scores 174 over a series of assessments. The comparative progression indicator 172 may also graphically illustrate the most recent subindices and the indices over time and/or trends in subindices (e.g., moving average).

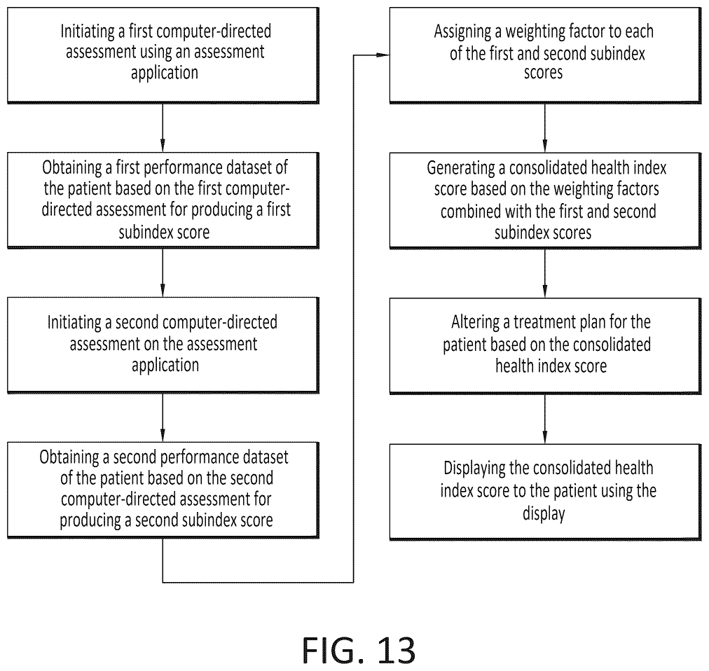

FIG. 13 provides an exemplary flow chart for how the method of enhancing patient education is achieved by generating consolidated health index score 128. As depicted, the present disclosure relates to a computer-implemented method comprising the steps of (1) initiating a first computer-directed assessment 136 on the assessment application 112, (2) with a tracking device 104, 106 communicatively connected to the computing unit 102, obtaining a first performance dataset of the individual 101 based on the first computer-directed assessment 136 for producing a first subindex score 130, (3) initiating a second computer-directed assessment 138 on the assessment application 112, (4) with the tracking device 104, 106 communicatively connected to the computing unit 102, obtaining a second performance dataset of the individual 101 based on the second computer-directed assessment 138 for producing a second subindex score 132, (5) assigning a weighting factor to each of the first and second subindex scores 130, 132, and (6) generating a consolidated health index score 128 based on weighting factors and combined first and second subindex scores 130, 132, and (7) displaying the consolidated health index score 128 to the patient 101 using the display 122. The subindices and consolidated health index enhance patient education by providing healthcare providers with a tool to simplify and present to their patients in a comprehensible way the status of their condition, areas for improvement, reasoning for targeted treatment, and their progression. The method further includes a step for altering a treatment plan 166 for the patient 101 based on the consolidated health index score 128. In an embodiment, the treatment plan may also be altered based on the subindex scores or individual subindex metrics or sub-subindices. While the method depicted in FIG. 13 depicts only first and second subindex scores 130, 132, the consolidated health index score 128 is generated by more than one subindex score, i.e., first to n^{th} subindex scores or first to n^{th} computer-directed assessments.

The disclosed system 100 adds versatility to rehabilitation for both impaired individuals (e.g., patients) and providers. The system 100 advantageously offers remote capability to accommodate telemedicine rehabilitation applications. Patients may carry out clinically verified assessments in the comfort of their own homes. This, in turn, reduces the number of face-to-face clinic visits needed to ensure therapeutic effectiveness. The system 100 also adds versatility for the patient by reducing the cost associated with outpatient physical therapy. The cloud storage capability (e.g., network 124) allows clinicians to remotely access patient data, easily review sequential therapy sessions to assess patient progression or regression, and modify the therapy regimens in response to patient performance. This versatility for patients to perform their therapy at home and the ability for clinicians to track these at-home therapy sessions may also improve patient compliance. The regular, consistent movement of an impaired joint in a controlled manner leads to better patient outcomes. Thus, when patients perform their therapy more frequently (i.e., consequently from implementing a simplified, accessible system 100 with enhanced patient education) there is an increased likelihood that their therapy will remediate their impairment and relieve their pain.

It is to be understood that even though numerous characteristics and advantages of various embodiments of the present disclosure have been outlined in the foregoing description, together with details of the structure and function of various embodiments thereof, this detailed description is illustrative only, and changes may be made in detail, especially in matters of structure and arrangements of parts within the principles of the present disclosure to the full extent indicated by the broad general meaning of the terms in which the appended claims are expressed.

1. A system for generating a consolidated health index score based on physical performance by an individual, the system comprising:
a first computing unit having an assessment application, a processor, one or more hardware storage devices, and a display, wherein the assessment application includes one or more computer-directed assessments;
a tracking device communicatively connected to the first computing unit and configured to observe physical assessment performance of the individual during the one or more computer-directed assessments;
wherein the one or more hardware storage devices store instructions that are executable by the system to:
initiate a first computer-directed assessment on the assessment application arranged to be completed using the tracking device;
obtain a first performance dataset of the individual from the tracking device and based on the first computer-directed assessment for producing a first subindex score;
initiate a second computer-directed assessment on the assessment application arranged to be completed using the tracking device;
obtain a second performance dataset of the individual from the tracking device and based on the second computer-directed assessment for producing a second subindex score;
assign a weighting factor to each of the first and second subindex scores; and
generate the consolidated health index score for presentation on the display based on weighting factors and combined first and second subindex scores.

2. The system according to claim 1, wherein the first computer-directed assessment generates and evaluates a first metric or set of metrics quantifying at least one of range of motion, proprioception, balance, sensorimotor control, neuromuscular control, strength, oculomotor control, coordination, vestibular function, reaction time, endurance, or cognition.

3. The system according to claim 2, wherein the second computer-directed assessment generates and evaluates a second metric or set of metrics different from the first metric.

4. The system according to claim 1, wherein the first subindex score is based on subjective and/or objective data.

5. The system according to claim 1, wherein the tracking device comprises at least one of:
a camera,
a lidar sensor, and
an inertial measurement unit (IMU).

6. The system according to claim 1, wherein the assessment application is configured to produce a collective index scoring report including the first and second subindex scores and the consolidated health index score.

7. The system according to claim 3, wherein the instructions stored by the one or more hardware storage devices are further executable by the system to:
initiate a third computer-directed assessment on the assessment application;
obtain a third performance dataset of the individual based on the third computer-directed assessment for producing a third subindex score;
assign an additional weighting factor to the third subindex scores; and
generate the consolidated health index score for presentation on the display based on weighting factors and combined first, second, and third subindex scores.

8. The system according to claim 7, wherein the third computer-directed assessment evaluates a third metric or set of metrics different from the first and second metrics.

9. The system according to claim 1, wherein the first and second subindex scores are produced using historical performance datasets stored in a collective database and factored into a dynamic calculation with the first and second performance datasets of the individual, the collective database being communicatively connected to the first computing unit.

10. The system according to claim 1, wherein the assessment application includes a machine learning algorithm to automatically recommend one or more rehabilitation or exercise strategies based on metric and index analysis.

11. The system according to claim 10, wherein the assessment application is arranged to dynamically update the one or more rehabilitation or exercise strategies based on the performance of the individual during and or after completion of the first and second computer-directed assessments.

12. A method of generating a consolidated health index score based on physical assessment performance by an individual using a tracking device communicatively connected to a computing unit having an assessment application, a processor, one or more hardware storage devices, and a display, wherein the assessment application includes one or more computer-directed assessments, wherein the computing unit is arranged to record physical performance of the individual with the tracking device during the one or more computer-directed assessments, the method comprising:
initiating a first computer-directed assessment on the assessment application and arranged to be completed with the tracking device;
obtaining a first performance dataset of the individual from the tracking device based on the first computer-directed assessment for producing a first subindex score;
initiating a second computer-directed assessment on the assessment application arranged to be completed using the tracking device;
obtaining a second performance dataset of the individual from the tracking device and based on the second computer-directed assessment for producing a second subindex score;
assigning a weighting factor to each of the first and second subindex scores; and
generating the consolidated health index score for presentation on the display based on weighting factors and combined first and second subindex scores.

13. The method of claim 12, further comprising the step of evaluating a first metric or set of metrics, wherein the second computer-directed assessment evaluates a second metric or set of metrics different from the first metric.

14. The method of claim 12, wherein the step of obtaining a first performance dataset of the individual includes monitoring physical performance of the individual using a tracking device attached to or associated with the individual, the tracking device being communicatively connected to the first computing unit.

15. The method of claim 12, wherein the step of obtaining a first performance dataset of the individual includes monitoring physical performance of the individual using a camera communicatively connected to the first computing unit.

16. The method of claim 12, further comprising the step of producing a collective index scoring report comprising:
the first and second subindex scores;
the consolidated health index score;
wherein the assessment application is arranged to dynamically adjust based on the physical performance of the individual during and or after completion of the first and second computer-directed assessments.

17. The method of claim 12, further comprising:
initiating a third computer-directed assessment on the assessment application;
obtaining a third performance dataset of the individual based on the third computer-directed assessment for producing a third subindex score;
assigning an additional weighting factor to the third subindex scores; and
generating the consolidated health index score based on weighting factors and combined first, second, and third subindex scores.

18. The method of claim 12, wherein the steps of obtaining first and second subindex scores include factoring historical performance datasets from a collective database into a dynamic calculation with the first and second performance datasets of the individual.

19. The method of claim 12, wherein the subindex is calculated based on a first metric or set of metrics, at least one of the first and second computer-directed assessments, patient characteristics, or a comparison of population used.

20. A method of enhancing patient education based on physical performance by a patient using a tracking device communicatively connected to a computing unit having an assessment application, a processor, one or more hardware storage devices, and a display, the method comprising:
initiating a first computer-directed assessment on the assessment application and arranged to be completed with the tracking device;
obtaining a first performance dataset of the patient from the tracking device based on the first computer-directed assessment for producing a first subindex score;
initiating a second computer-directed assessment on the assessment application and arranged to be completed with the tracking device;
obtaining a second performance dataset of the patient from the tracking device and based on the second computer-directed assessment for producing a second subindex score;
assigning a weighting factor to each of the first and second subindex scores; and
generating a consolidated health index score based on weighting factors combined with first and second subindex scores;
displaying the consolidated health index score or subindex scores represented on one or more bounded scales to the patient using the display;
wherein the steps of producing the first subindex score and the second subindex score includes generating a metric or set of metrics that quantifies at least one of range of motion, proprioception, balance, sensorimotor control, neuromuscular control, strength, oculomotor control, coordination, vestibular function, reaction time, endurance, and cognition, and where the metric or set of metrics are represented on one or more bounded scales.
